(19) United States
(12) Patent Application Publication
(10) Pub. No.: US 2003/0113939 A1
(43) Pub. Date: Jun. 19, 2003

(54) Modified surface for carrying out or detecting affinity reactions

(75) Inventors: Gunter Gauglitz, Tuebingen (DE); Andreas Brecht, Lenningen (DE); Jacob Piehler, Tuebingen (DE)

Correspondence Address: KALOW & SPRINGUT LLP, 488 MADISON AVENUE, 19TH FLOOR, NEW YORK, NY, 10022, US

(73) Assignee: Bodenseewerk Perkin-Elmer GMBH, Foster City, CA (US)

(21) Appl. No.: 10/236,862

(22) Filed: Sep. 6, 2002

Related U.S. Application Data
(63) Continuation of application No. 09/673,343, filed on Jan. 22, 2000, now abandoned.
Application No. 09/673,343 is a 371 of international application No. PCT/EP99/02485, filed on Apr. 13, 1999.

(30) Foreign Application Priority Data
Apr. 15, 1998 (DE) DE19816604.4

(51) Int. Cl. G01N 33/543; C12M 1/34; B05D 3/00

(52) U.S. Cl. 436/518; 435/287.2; 427/2.11

(57) ABSTRACT

A device for carrying out and/or detecting affinity reactions comprises at least one surface or boundary surface on which an affinity reaction can take place, and at least one polymer bound to the surface. According to the invention, this device is obtainable by bonding the polymer to the surface in its pure phase, i e. essentially free of solvents. The polymer is preferably a terminally homo- or heterosubstituted bifunctional polymer, a corresponding polyalkylene glycol, in particular a polyethylene glycol, being preferred. The surface can be made of a metal oxide, in particular silicon dioxide. Before bonding the polymer, an activation of the surface, preferably by introducing functional groups by means of a silanization, can be effected.

### CROSS REFERENCE TO RELATED APPLICATIONS

This application is a continuation of application Ser. No. 09/673,343, filed Jan. 22, 2000, which is a 371 of international application Ser. No. PCT/EP99/02485, filed Apr. 13, 1999, which claims priority from DE19816604.4, filed Apr. 15, 1998, which are all incorporated herein by reference.

The invention concerns a device for carrying out and/or detecting affinity reactions, in particular a probe device, having at least one surface or boundary surface on which an affinity reaction takes place and at least one polymer bound to the surface.

The background of the invention is among others the aspiration to detect affinity reactions, in particular biochemical affinity reactions with optical, acoustic or other physical methods, without using corresponding marker substances. For this purpose, in particular optical processes, such as the surface plasmon resonance, the reflectometric interference spectroscopy, the frustrated total reflection, grating couplers and the so-called integrated-optical processes, are suited as physical detecting method.

The prerequisite for such a detection without using markers is the selective interaction of the molecules, in particular the biomolecules, to the surface or boundary surface of a physical signal transducer. For this purpose, one of the interacting substances has to be immobilised at the surface in a defined form. This attachment has to be sufficiently stable in order to guarantee a long durability, in particular also under unfavourable conditions, such as chemically aggressive conditions. Moreover, it has to be ensured that no molecules are nonspecifically adsorbed to the surface, but that specifically only those molecules are adsorbed, of which the interaction with the attached substances is to be detected. In a detection without using markers, this requirement is especially important as the detecting methods used in this case detect any variation at the surface and can therefore not differentiate between a specific and a nonspecific bond.

Usual signal transducer surfaces which consist e.g. of gold, quartz or metal oxides cannot meet the mentioned requirements. Therefore, it is already known to purposely modify such surfaces. In the process, it has to be ensured, however, that such a modification has no negative influence on the detection itself, e.g. on the sensitivity thereof. With optical detecting processes, usually very thin surface layers, preferably <50 nm, having a uniform design and a high density, are therefore aimed for. This is in particular true for optical detecting processes which are based on directed reflection, such as interferometric or ellipsometric processes, but also for a total reflection if the penetration depth of the evanescent field is low and therefore the sensitivity is largely reduced with the distance from the surface, e.g. in case of the grating coupler.

Thus, linear long-chained alkyl groups can be covalently attached on gold or quartz, at the same time forming a monolayer, and be used for immobilising biochemical components, cf. e g. J. Rickert et al. in Biosensors & Bioelectronics Vol. 11, No. 6/7, pp 591-598, 1996 In this case, however, a passive effect is concerned which can only be purposefully influenced with difficulty. Here, the screening is purely effected by covering, at the same time forming a thick layer of chains which interact. These are called self-assembling monolayers (SAM).

Furthermore, the attachment of polyethylene glycol and other polymers at surfaces has already been examined with respect to their suitability for immobilising chemical and biochemical substances. The publication by J. Piehler et al. in Biosensors & Bioelectronics Vol. 11, No. 6/7, pp. 579-590, 1996, shows, however, that no efficient screening of the surface can be achieved. This is probably due to a too low density of the covalently attached polymer chains.

Therefore, in the field of the detection of affinity reactions without using markers, the covalent attachment of dextran layers with hydrogel properties is still widely used (see S. Löfas in Pure Appl. Chem. 67 (1995), 829-834). It is true that in such a surface modification the high hydrophily of the dextran polymers provides for a low adsorption of proteins and thus for a normally low nonspecific bond. However, a screening against the nonspecific bond of macromolecules is not principally guaranteed. Moreover, a modification of the surface with dextran layers has the disadvantage that the attachment of the immobilised component is not effected at a surface in a defined way, but statistically within the hydrogel matrix having a diameter of usually 50 to 200 nm. Thus, the accessibility of the immobilised component for the substance interacting therewith is not defined and a considerable influence on the interaction can be noticed.

Therefore, the object of the invention is to avoid the disadvantages known from the prior art. In particular, a modified surface is to be provided for carrying out and/or detecting affinity reactions on which nonspecific interactions are efficiently suppressed. The substances immobilised at the surface are to be stably attached or attachable in the form of thin layers, at the same time having a good accessibility, such that such modified surfaces comprise a long durability and a good regenerating capacity. The surfaces are to be suitable in a particular manner for a detection of affinity reactions, in particular biochemical affinity reactions, with optical detection methods without using markers

This object is achieved by the device having the features of claim 1 and the process having the features of claim 19. Preferred embodiments of this device and this process are presented in the dependent claims 2 to 18 and 20 to 23, respectively. By referring to all claims, the wording thereof is thus made to be the contents of this description.

According to the invention, the above mentioned device can be achieved in that the bonding of the polymer to the surface is effected in its pure phase, i.e. essentially free of solvents. By the bonding in a pure phase, a modified surface with special properties can be obviously provided which makes the same particularly suitable for carrying out or for detecting affinity reactions compared to known surfaces. The special properties of the surface modified according to the invention can be put down to the fact that mutually repelling polymer chains form a very thin surface coating of a high density and orientation (formation of a dense polymer “lawn” of juxtaposed polymer molecules which optionally repel each other). This results in an effective screening against the nonspecific bond of any molecules. Thus, the modified surface is suited in a particular way for an immobilisation/attachment of a (biochemical) substance and thus for carrying out or detecting a corresponding affinity reaction with this substance. Such a modified surface can obviously not be realised when using solvents for the attachment.

The device according to the invention can in particular be a probe device with a signal transducer surface mentioned at the beginning. Such devices are also known as so-called biosensors. The expression “bond” in the sense of the invention is usually a covalent bond.

The polymer bound to the surface is in particular a polymer which is to a great extent linear, preferably comprising only a short chain. When using linear polymers, one can achieve a particularly dense arrangement of the polymer chains side by side. By means of short chains, one can produce thin layers

According to the invention, it is preferred for the described polymer to be a so-called hydrophilous polymer. Such polymers, of which examples are given below, can repel macromolecules in an aqueous medium due to their excluded volume. This basically advantageous property can be shown to its best advantage in the invention.

The molar mass of the polymers can be principally freely chosen, however, it is preferably between 200 and 50,000 g/mol, more preferably between 200 and 5,000 g/mol. In view of the not unambiguously defined boundary between oligomers and polymers, polymers having a low molar mass can be also described as oligomers in the sense of the invention. Within the mentioned preferred regions of molar masses, molar masses between 200 and 20,000 g/mol and within this region molar masses between 500 and 5,000 g/mol are to be emphasised. Chain lengths of the straight polymer of 2 to 20 nm, preferably 2 to 10 nm, can correspond to the mentioned polymer masses.

Furthermore, in the invention the polymer has in particular a melting point of <200° C., preferably of <100° C. As already described, in the invention the bonding of the polymer to the surface is effected in its pure phase. Therefore, it is preferred for the polymer to be present in a liquid form or to be easily converted into the liquid phase. This is preferably effected by melting, such that a polymer meltable at comparably low temperatures (<200° C., better <100° C.) is particularly well suited for the invention.

In a further development, the polymer has terminal functional groups, and principally monofunctionalised polymers can be employed. Preferably, the polymers are, however, terminally homo- or heterosubstituted bifunctional polymers, such that one terminal function is provided for bonding the polymer to the surface and the other terminal function is provided for bonding an interactive substance (ligand).

The functional groups present at the polymer can be NH₂, OH, SH, COOH, OCH₃, oxirane, SCN and/or OCN groups, NH₂, OH and/or COOH groups being preferred.

According to the invention, mixtures of various polymers or even copolymers can be easily used. Such mixtures can be for example mixtures of polymers which only differ in their molar mass, or mixtures of polymers which carry different functional groups. In the latter case, optionally various functional groups for the attachment of interactive substances are provided.

In preferred embodiments of the invention, the polymer is a polyalkylene glycol. Among the typical representatives of the polyalkylene glycols, namely polymethylene glycol, polyethylene glycol and polypropylene glycol, the use of polyethylene glycol is preferred. As preferred polyethylene glycols (PEG), the homosubstituted bifunctional substances diamino-PEG and dicarboxy-PEG can be mentioned, wherein molar masses between 200 g/mol and 20,000 g/mol, preferably molar masses of about 2,000 g/mol and 20,000 g/mol, in particular between 2,000 and 5,000 g/mol, are preferred. Naturally, copolymerides of various polyalkylene glycols or mixtures of various polyalkylene glycols can also be used.

Though with the mentioned polyalkylene glycols especially good results are achieved in the invention, the invention is not restricted to the use of these polymers. Basically, all polymers which can be converted into their pure phase can be used, in particular the hydrophilous polymers having a high excluded volume and a corresponding functionalisation. As particularly suitable polymers one can mention the polyalkylene imines, in particular the polyethylene imine being emphasised Further examples are terminally functionalised polyvinyl alcohols, polyhydroxyethyl methacrylates and polyamides.

As already shown, the surface on which the polymer is bound in the invention, is situated at a suitable carrier or a suitable substrate forming the device or part of the device. Preferably, the surface is formed at a signal transducer or biosensor In this case, the surface is preferably planar.

Before bonding the polymer, the surface can comprise free OH groups in the invention, as such groups form a good base for a bonding.

The surface or the carrier itself can be made of a metal, for example gold and/or silver, or a metal oxide, which is preferably silicon dioxide. As further preferred surface/carrier materials, one can also mention glass, titanium dioxide (TiO₂), tantalum oxide (Ta₂O₅) and indium tin oxide (ITO).

In further preferred embodiments of the invention, the-surface is modified/activated before bonding the polymer, preferably functional groups being introduced during this activation. These functional groups can then directly or by means of additional coupling reagents be converted with functional groups of the polymer and thus the polymer can be attached. The introduction of functional groups to the surface is appropriate when the surface itself does not allow a satisfactory conversion with the polymer. When metals are used as surface/carrier, the functionalisation can be effected by alkyl thioles. These are preferably alkyl thioles of the general formulae HS—(CH₂)ₙY or (S—(CH₂)ₙY)₂ with a suitable functional group, in particular an epoxy group as group Y and with n between 1 and 15. With metal oxides, the introduction of the functional groups to the surface is in particular effected by silanization, wherein preferably silane compounds of the general formula X—Si—R—Y can be used. Such silane compounds are covalently bound to the surface via the group named X. The residual X can preferably be a residual alkoxy or a residual halogen, in particular a residual chlorine. The group R is usually an alkyl chain which is preferably linear and can be interrupted by hetero atoms. The chain length of the group R can usually be between 4 and 30 atoms. The group Y is a suitable functional group, preferably an epoxy group.

The introduced functional groups can preferably be NH₂, OH, SH, ester, oxirane, SCN and/or OCN groups. When using ester groups, so-called activated esters, i.e. particular reactive ester groups, are preferred.

As already mentioned, the functional groups introduced to the surface are usually converted with functional groups of the polymer for attaching the same. From the following list, one can see particularly suited combinations of the functional groups of such reaction partners,

| Functional group at the surface | Functional group at the polymer |
|---|---|
| Oxirane, active ester, SCN, OCN group | NH₂, OH, SH group |
| oxirane group | COOH group |
| NH₂, OH, SH group | oxirane, SCN, OCN group |
| OH group (activated with divinyl | OH group |
| sulfone, carbonyidiimidazole) | |
| OH group (activated with tosyl chloride, | NH₂ group |
| tresyl chloride, cyanur chloride, divinyl | |
| sulfone | |

As already described, according to the invention the polymer is preferably used as liquid or molten polymer to be bound to the surface. In this case, according to the invention a decomposition of the components involved in the reaction does not yet take place. Correspondingly, polymers with a melting point <200° C., preferably <100° C. are preferred. Usual and preferred temperatures for bonding the polymer to the surface are correspondingly between approximately 50° C. and approximately 100° C., and within this region temperatures between 50° C. and 80°°C. are to be emphasised. A usual conversion temperature is for example approximately 60° C.

In order to achieve a bonding of the polymer to the surface in its pure phase, longer reaction times can be preferred. Thus, the conversion can be effected within a period of some hours to some (several) days. Usual conversion times are between 12 and 60 hours. In order to achieve a reliable bonding, it is often practical and preferred in case of a lower reactivity of the components to choose higher reaction temperatures and/or longer reaction times. This is in particular promising when as functional groups at the surface as well as at the polymer OH groups and COOH groups are chosen.

The device according to the invention is further characterised in that at the polymer bound to the surface a ligand, in particular a biochemically active ligand, is immobilised. This ligand usually coupled to the polymer via the second functional group is then available for the actual affinity reaction, in which the component to be detected interacts with the immobilised ligand. The immobilisation of the ligand can be effected in the usual manner by means of usual reactions, as will be further illustrated below by means of examples.

The invention furthermore comprises a surface coating for surfaces and carriers which are employed for carrying out or detecting affinity reactions. The features of such a surface coating can be taken from the text of the description above.

As already described, the surface or surface coating, respectively, modified according to the invention, has decisive advantages with respect to the prior art. Thus, by bonding the polymer to the surface in its pure phase, a dense and tightly bound thin layer having a high stability is provided which enables a highly specified bonding of the species to be detected or converted, respectively, after the immobilisation of a suitable ligand A nonspecific bonding of other substances is widely excluded. The modified surface has a long durability and a good regeneration capacity. All these advantageous properties make the modified surface particularly suitable for the use in biosensors or bioprobes. Biochemical interactions at boundary surfaces can be detected by means of the already mentioned methods, in particular the optical methods, without using markers.

Furthermore, the invention comprises a process for manufacturing a device with a modified surface being provided for carrying out or detecting affinity reactions. Here, at least one polymer is bound to a surface on which an affinity reaction is to be effected in its pure phase, i.e. essentially without using a solvent. With respect to the preferably used polymers, the preferably used surfaces and the preferably used process parameter, reference is made to the above description.

Finally, the invention comprises the use of the described device for detecting affinity reactions, in particular biochemical affinity reactions, without using markers. As a detection process, here preferably an optical process, in particular the so-called reflectometric interference spectroscopie, is used.

The described features and further features of the invention can be taken from the following description of preferred embodiments in connection with the subclaims, examples and drawings. Here, the individual features can be realised separately or in combination of several features. The drawings show:

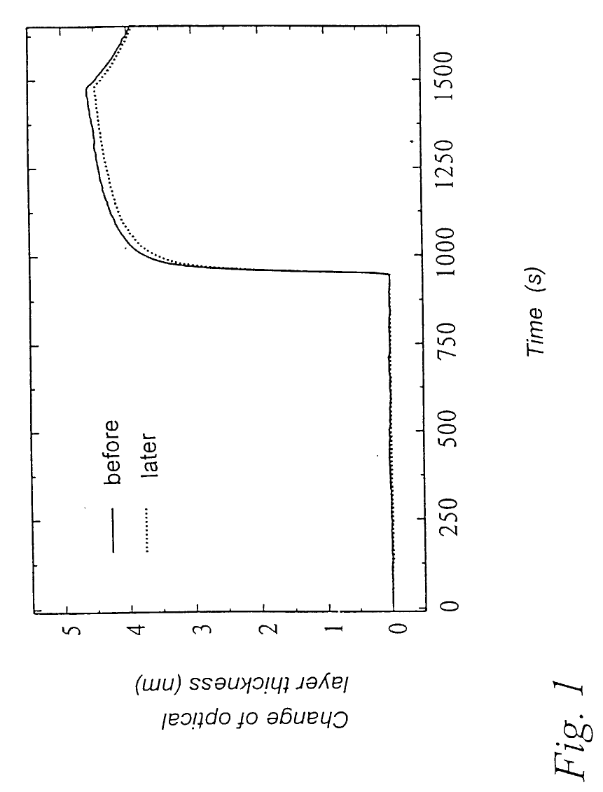

FIG. 1 the variation in time of the optical layer thickness when examining a first surface modified according to the invention for specific and nonspecific interaction,

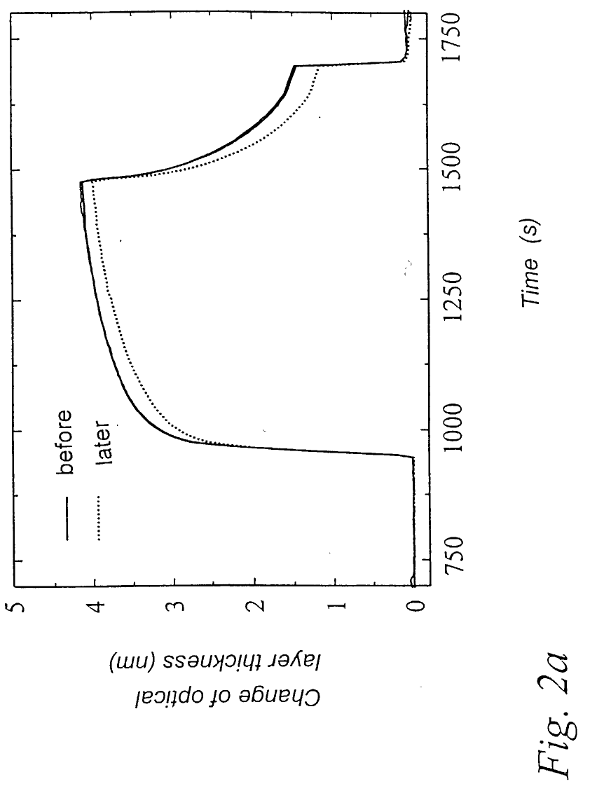

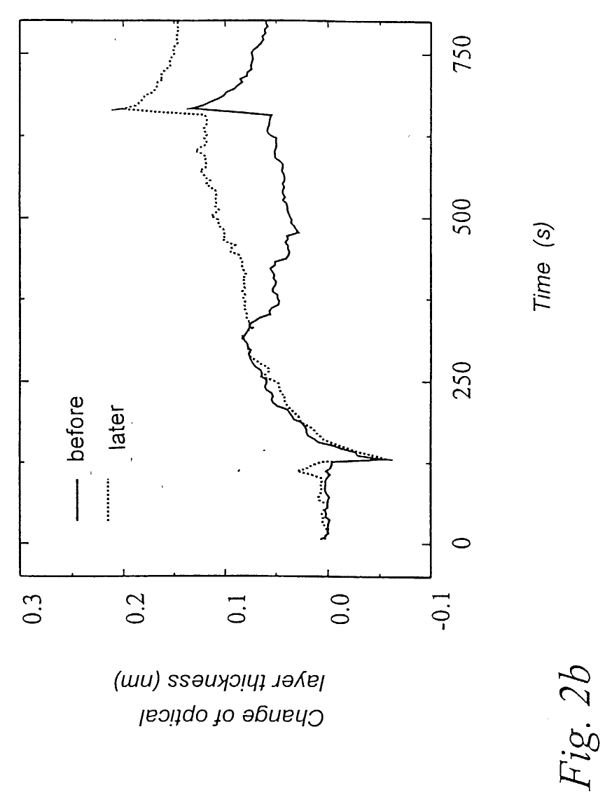

FIG. 2a the variation in time of the optical layer thickness when examining a second surface modified according to the invention for nonspecific interaction,

FIG. 2b the variation in time of the optical layer thickness when examining a second surface modified according to the invention for specific interaction, and

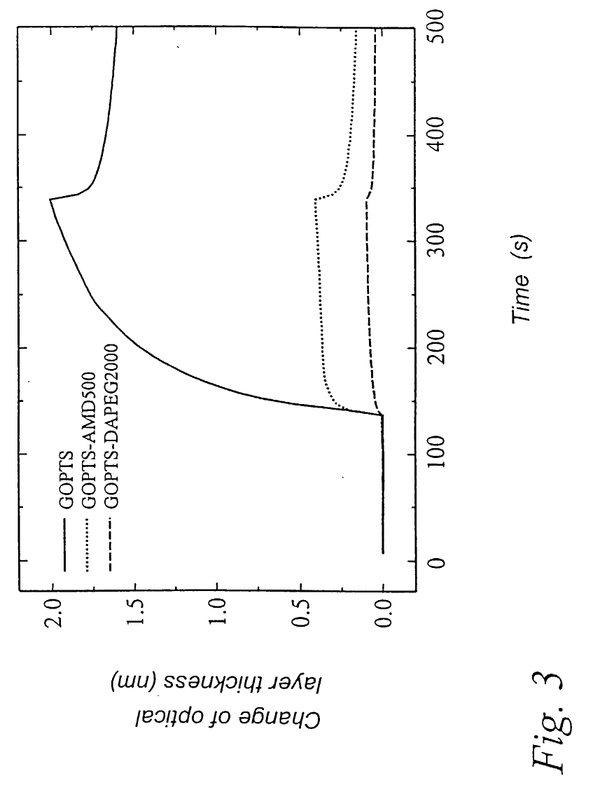

FIG. 3 the variation in time of the optical layer thickness when examining a surface modified according to the invention and two reference surfaces for nonspecific interaction.

### EXAMPLE 1

For producing a modified surface, a glass carrier is taken onto which an SiO₂-layer with a thickness of 300 to 400 nm has been deposited by a sputtering process. This glass carrier with the SiO₂-layer was firstly treated as follows:

rubbing off coarse impurities with a paper towel,

incubation with 6 N NaOH for 2 min and subsequently washing off with water,

incubation with a freshly produced mixture of conc. H₂SO₄ and conc. H₂O₂ (3:2) for 30 min and subsequently washing off with water, and

drying the surface at room temperature in air.

The thus pre-treated surface was subsequently activated with glycidyloxipropyltri-methoxysilane (GOPTS), received from Fluka, Deisenhofen. In the process, the following procedure was followed:

incubation of 10 μl/cm² of pure GOPTS on the surface for 1 hour at room temperature, and

subsequently washing off with dry acetone and drying with nitrogen.

Directly after the activation of the surface by silanization with GOPTS, a bifunctional polyethylene glycol (PEG), namely a diamino-PEG with a molar mass of 2,000 g/mol (product DAPEG 2000 from the company Rapp Polymere, Tübingen), was bound to the activated surface. The conversion was effected as follows:

depositing 5 mg/cm³ of polyethylene glycol onto the surface and melting the polyethylene glycol at approx. 60° C. to approx. 70° C. in an oven,

placing a glass plate onto the surface for evenly distributing the molten polymer,

temperature treatment/conversion in the oven at about 60° C. for 36 hours, and

washing off the excess polyethylene glycol with water and drying in air at room temperature.

After this conversion, the polyethylene glycol (Polymer) is covalently bound or coupled, respectively, to the surface. The second amino group of the bifunctional polyethylene glycol is available for the immobilisation of a chemical or biochemical component, which in turn can take part in an affinity reaction.

In the present case, a carboxyl-functionalised triazine derivate, namely 4-chloro-6-(isopropylamino)-1,3,5-triazine-2-(6′-amino)-capron acid (CTCA) was coupled to the terminal free amino group. The following procedure was followed:

adding 1 μl of diisopropylcarbodiimide (DIC) to 10 μl of a solution of 1 mg CTCA per 10 μl of dimethylformamide (DMF), (DIC and DMF from Fluka, Deisenhofen),

incubation of 5 μl/cm² of the thus produced solution on the surface for at least 6 hours, and

subsequently washing off the surface with DMF and water and drying at room temperature in air.

For the functional characterisation of the modified surface with the immobilised ligand CTCA, the nonspecific and the specific bond of proteins was examined by means of the reflectometric interference spectroscopy (RifS). In the process, the following solutions were used for the incubation:

a. 1 mg/ml of ovalbumin,

b. 100 μl/ml of calf serum, and

c. 50 μg/ml of a protein specific for CTCA, namely anti-simazine antibodies, fab-fragment.

The reflectometric interference spectroscopy (RifS) is known as a detection process for the detection of affinity reactions without using markers. The basic factors of the process are for example described in the publication of G. Gauglitz et al., in Sens. & Act., B 11, 21 to 27 (1993). The interference of white light which is reflected at thin layers is determined. The bonding of proteins to the surface increases the optical layer thickness of the film which is detected as variation in the reflection spectrum (A. Brecht et al., Anal. Chim. Acta, 311, 289 to 300 (1995)). Details of the equipment used in the present case can be taken from the publication of J. Piehler et al. in Biosensors & Bioelectronics Vol. 11, No. 6/7, 579-590, 1996, which was already mentioned in the beginning.

The essential results of the functional characterisation of the modified surface prepared according to example 1 are shown in FIG. 1. Here, the variation of the optical layer thickness is plotted in dependence on the time. Moreover, the stability of the layers was examined by determining the functional properties after 100 regenerations of the surface with 0.1 M HCL.

FIG. 1 on the one hand shows that in an incubation with ovalbumin no significant nonspecific adsorptions of this protein could be detected. The period of the ovalbumin incubation is presented in FIG. 1 by the corresponding time bar.

In the incubation with calf serum, a low nonspecific bond of 100 to 200 pg/mm² was found which is negligible and not shown in FIG. 1 for reasons of clarity.

Furthermore, FIG. 1 shows that the specific bond of anti-simazine fab to the immobilised triazine derivate can be very clearly detected. Here, the specific bond of about 4 to 5 ng/mm² fab corresponds to the amount which is to be expected for a monolayer of this protein. One can also clearly see that the functional properties of the modified surface are maintained to a great extent in the course of 100 regenerations The specific bond is reduced by less than 5% after 100 regenerations

The illustrated advantages of the invention are thus convincingly confirmed by example 1.

For physically characterising the modified surface, in parallel the loading of the surface with the polymer was characterised by means of the spectral ellipsometry The measurements with this equally basically known detection method was effected in air. For reasons of the methodology, in this case silicon wafers oxidised with an SiO₂-layer (Wacker-Chemitronic, Burghausen) were used instead of the glass substrate.

The ellipsometric characterisation showed a surface loading of almost 4 ng/mm² in the case of example 1. This value is above the loading to be achieved by other methods by a factor of 2 to 4. It corresponds to a surface concentration of more than 10¹² polymer chains/mm² or a surface of less than 1 nm² per polymer chain. This shows the dense packing of the polymer chains on the surface in the invention.

### EXAMPLE 2

In the procedure described in Example 1, a glass substrate is pre-treated with an SiO₂-layer, silanized and converted with polymer. Only instead of the diamino-PEG, another bifunctional polyethylene glycol, namely dicarboxy-PEG having a molar mass of 2,000 g/mol (product DCPEG 2000 from the company Rapp Polymere, Tübingen) is used.

Subsequently, an amino-functionalised thrombine inhibitor (TI) (BASF AG, Ludwigs-hafen) is coupled to the remaining free carboxyl groups. The following proceeding is carried out for doing so

addition of 1 μl of DIC to 10 μl of a solution of 2 mg of thrombine inhibitor per 10 μl of DMF,

incubation of 5 μl/cm² of the thus prepared solution on the surface for at least 6 hours, and

subsequently washing off the surface with DMF and water and drying at room temperature in air.

In Example 2, as well, the functional and the physical characterisation of the modified surface is effected in the manner already described in Example 1. The results are shown in FIGS. 2a and 2b, the variation in the optical layer thickness being in each case plotted in relation to the time.

FIG. 2a shows that in an incubation with calf serum a very weak nonspecific bond of 100 to 200 pg/mm² can be established. The duration of the incubation is here, too, shown by the time bar. An essential influence of this nonspecific bond after 100 regenerations with 0.1 M HCL cannot be detected.

In contrast thereto, from FIG. 2b one can clearly see that the specific bond of thrombine as specific protein is clearly detectable for the immobilised thrombine inhibitor via the variation in the optical layer thickness. Here, too, the obtained value indicates the formation of a monolayer. In case of the incubation with ovalbumin, in turn no significant nonspecific bond to the surface could be detected. The latter is not shown in FIG. 2b for reasons of clarity.

The examination of the stability of the functional properties after 100 regenerations with 0.1 M HCL gives a result comparable to that of Example 1, namely that the specific bond is reduced by less than 5%.

The results for the surface loading obtained at an oxidised silicon wafer with spectral ellipsometry, too, correspond to the results of Example 1.

### EXAMPLE 3

The specific bond and the nonspecific bond are examined with various polyethylene glycols (PEGs).

In addition to the two polyethylene glycols used in Examples 1 and 2, the following polymers are used:

methoxyamino-PEG with a molar mass of 2,000 g/mol (product MAPEG 2000 from the company Rapp Polymere, Tübingen),

diamino-PEG with a molar mass of 20,000 g/mol (product DAPEG 20000 from the company Rapp Polymere, Tübingen), and

dihydroxy-PEG with a molar mass of 3,000 g/mol (product DHPEG 3000 from MERCK).

The preparation of the modified surfaces is effected by means of the five used polyethylene glycols as described in Examples 1 and 2. Subsequently, in an also already described manner, the nonspecific bond of ovalbumin and calf serum as well as (partially) the specific bond are examined, including the results of Examples 1 and 2. For a comparison, the nonspecific bond at a pure GOPTS-layer is additionally taken into consideration.

The results of the examinations are summarised in the following table:

| | | Ovalbumin | calf serum | max. specific |
|---|---|---|---|---|
| Polyethylene | | (1 mg/ml) in | (100 μl/ml) in | bond in |
| glycol | Ligand | (pg/mm²) | (pg/mm²) | (ng/mm²) |
| DAPEG 2000 | CTCA | <10 | 100-200 | 4-5 |
| DAPEG 2000 | — | <10 | 100-200 | — |
| DAPEG 20000 | CTCA | <100 | 150-200 | 2,5 |
| DHPEG 3000 | — | <10 | 150-200 | — |
| MAPEG 2000 | — | <10 | <100 | — |
| DCPEG 2000 | — | <10 | 100-200 | — |
| DCPEG 2000 | TI | <10 | 100-200 | 4-5 |
| GOPTS 2000 | — | 100-300 | ab. 2000 | — |

The results of the table show that in comparison with a pure GOPTS-layer in a use of the polyethylene glycols according to the invention, in all cases a low nonspecific bond or interaction, respectively, can be achieved. With the results in case of a polymer with a molar mass of 20,000 g/mol one expects a somewhat higher nonspecific bond in case of higher molar masses. In the present test, the polyethylene glycol with a terminal methoxy function shows a somewhat lower nonspecific bond.

### EXAMPLE 4

The unspecific bond from a soluble bacteria fraction at a surface modified according to the invention is compared to two reference surfaces. Here, as reference surfaces a silanized surface and a surface with polymer hydrogel on a dextran basis (extent about 40 nm) are chosen.

For preparing the modified surfaces, the following procedure is followed:

1. The silanized surface is prepared as described in Example 1.

2. For the surface with polymer hydrogel on a dextran basis, first of all an amino dextran having a molar mass of 500,000 g/mol is prepared, as described in the already mentioned publication of J. Piehler et al. in Biosensors & Bioelectronics Vol. 11, No. 6/7, pp 579-590, 1996.

This amino dextran denominated AMD 500 is coupled to a GOPTS-modified surface prepared according to Example 1 as follows

solving 100 mg of AMD 500 in 300 μl water and adjusting the pH-value to 7.5,

pipetting 5 μl/cm² of the thus obtained solution onto the silanized surface and placing a glass plate thereon for an even distribution,

allow to stand for 12 hours, subsequently washing off with water and drying in air at room temperature.

3. For the preparation of the surface modified according to the invention, the procedure of Example 1 (use of DAPEG 2000) is followed.

The bacteria fraction used for the characterisation of the nonspecific bond of all three surfaces is prepared by taking up the soluble fraction of 500 ml E. coli cell culture (branch DH5) in 20 ml of a buffer and diluting it with a buffer to 1/50.

The examination results at the three surfaces are shown in FIG. 3. One can clearly see that the surface modified according to the invention comprises a significantly lower nonspecific bond (maximal about 100 pg/mm²) than the merely silanized surface (about 2 ng/mm²) and the surface modified with hydrogel (about 400 pg/mm²). Here, it has to be taken into consideration that the polymer used according to the invention comprises a considerably lower molar mass than the hydrogel on a dextran basis (2,000 g/mol compared to 500,000 g/mol) and correspondingly a considerably lower physical layer thickness than that with hydrogel.

Zeichnungsbeschriftung

FIG. 1: Variation in the optical layer thickness [nm]

Time [s]

im Diagramm: before, after 100 regenerations

ovalbumin

anti-simazine fab

FIG. 2a: Variation in the optical layer thickness [nm]

Time [s]

im Diagramm: before, afterwards

calf serum 100 μl/ml

FIG. 2b: Variation in the optical layer thickness [nm]

Time [s]

im Diagramm: before, afterwards

thrombine, 50 μg/ml

FIG. 3: Variation in the optical layer thickness [nm]

Time [s]

1. Device for carrying out and/or detecting affinity reactions, in particular probe device, with at least one surface or boundary surface on which an affinity reaction takes place, and at least one polymer bound to the surface, obtainable by the bonding of the polymer to the surface in its pure phase, i.e. essentially free of solvents.

2. Device according to claim 1, characterised in that the polymer is largely a linear, preferably short-warp, polymer

3. Device according to claim 1 or claim 2, characterised in that the polymer comprises a molar mass of 200 to 50,000 g/mol, preferably of 200 to 20,000 g/mol.

4. Device according to one of the preceding claims, characterised in that the polymer has a melting point of <200° C., preferably of <100° C.

5. Device according to one of the preceding claims, characterised in that the polymer has terminal functional groups, these groups being preferably terminally homo- or heterosubstituted bifunctional polymers.

6. Device according to claim 5, characterised in that the functional groups are NH₂, OH, SH, COOH, OCH₃, oxirane, SCN and/or OCN groups, NH₂, OH and/or COOH groups being preferred.

7. Device according to one of the preceding claims, characterised in that the polymers are mixtures of various polymers or copolymers.

8. Device according to one of the preceding claims, characterised in that the polymer is a polyalkylene glycol, in particular a polyethylene glycol.

9. Device according to one of claims 1 to 7, characterised in that the polymer is a polyalkylene imine, in particular a polyethylene imine.

10. Device according to one of the preceding claims, characterised in that the surface is formed at a carrier or substrate.

11. Device according to one of the preceding claims, characterised in that the surface carries free OH groups before the bonding of the polymer.

12. Device according to one of the preceding claims, characterised in that the surface is made of a metal or a metal oxide, the metal oxide being preferably a silicon dioxide.

13. Device according to one of the preceding claims, characterised in that the surface is modified or activated before the polymer is bound to it, preferably functional groups being introduced for the modification/activation, in particular by silanization.

14. Device according to claim 13, characterised in that the functional groups are NH₂, OH, SH, ester, oxirane, SCN and/or OCN groups.

15. Device according to one of the preceding claims, characterised in that the polymer is employed as a liquid or molten polymer for the bonding at the surface.

16. Device according to one of the preceding claims, characterised in that the bonding of the polymer at the surface is effected at temperatures below 200° C., preferably between 40° C. and 100° C., in particular between 50° C. and 80° C.

17. Device according to one of the preceding claims, characterised in that the bonding of the polymer at the surface is effected within a period of some hours to some days.

18. Device according to one of the preceding claims, characterised in that at the polymer bound to the surface a ligand, in particular a biochemically active ligand, is immobilised which is available for an affinity reaction.

19. Process for preparing a surface-modified device for carrying out and/or detecting affinity reactions, in particular for preparing a surface-modified probe, characterised in that at least one polymer in a pure phase, i.e. essentially free of solvents, is bound to a surface on which an affinity reaction takes place.

20. Process according to claim 19, characterised by the use of a polymer with at least one of the features of claims 2 to 9.

21. Process according to claim 19 or claim 20, characterised by the use of a surface with at least one of the features of claims 10 to 14.

22. Process according to one of claims 19 to 21, characterised in that it is carried out according to at least one feature of claims 15 to 17.

23. Process according to one of claims 19 to 22, characterised in that a ligand, in particular a biochemically reactive ligand, is coupled to the polymer.

24. Use of the device according to at least one of claims 1 to 18 for a detection of affinity reactions, in particular biochemical affinity reactions, without using markers.

25. Use according to claim 24, characterised in that for the detection an optical process, preferably the reflectometric interference spectroscopy, is employed.
